(19) United States
(12) Patent Application Publication
(10) Pub. No.: US 2008/0268490 A1
(43) Pub. Date: Oct. 30, 2008

(54) METHODS FOR IN VITRO GROWTH OF HAIR FOLLICLES

(75) Inventors: Rebecca MORRIS, New York, NY (US); Teresa DICOLANDREA, Wyoming, OH (US)

Correspondence Address: WilmerHale/Columbia University, 399 PARK AVENUE, NEW YORK, NY, 10022, US

(73) Assignees: THE TRUSTEES OF COLUMBIA UNIVERSITY IN THE CITY OF NEW YORK, New York, NY (US); The Procter & Gamble Company, Cincinnati, OH (US)

(21) Appl. No.: 12/020,193

(22) Filed: Jan. 25, 2008

Related U.S. Application Data
(63) Continuation of application No. 11/872,473, filed on Oct. 15, 2007.
Application No. 11/872,473 is a continuation-in-part of application No. PCT/US06/14420, filed on Apr. 13, 2006.
(60) Provisional application No. 60/671,571, filed on Apr. 15, 2005.

(51) Int. Cl. C12Q 1/02; C12N 5/02

(52) U.S. Cl. 435/29; 435/373

(57) ABSTRACT

The invention is directed to a chemically defined animal cell culture media, and methods for preparing such a medium, wherein the media are suitable for culturing epidermal cells, preferably human epidermal cells, including cells of the hair follicle. The invention further provides for methods of culturing epidermal cells, hair follicles, and skin explants in the media as well as uses of the cell cultures and explant cultures in screening assays.

This application is a continuation-in-part of International Application No. PCT/US06/014420 (International Publication No. WO 06/113629), which was filed on Apr. 13, 2006, which claims priority to U.S. Provisional Application No. 60/671,571, which was filed on Apr. 15, 2005. These applications are hereby incorporated by reference in their entireties.

The invention disclosed herein was made with U.S. Government support under NIH Grant No. CA 45293 from the NCI. Accordingly, the U.S. Government may have certain rights in this invention.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described herein.

### BACKGROUND OF THE INVENTION

The epidermis consists of multiple layers of epithelial cells, including keratinocytes. Epidermal keratinocytes can be terminally differentiated, characterized by stratification and desmosome formation, or they can be in a state of growth and proliferation. Keratinocyte stem cells represent a population of cells which can be mobilized to reepithelialize the epidermis or to regenerate the hair follicle. Other follicular cell types include sheath cells, which form the follicular connective tissue, and the follicular papillae cells, which are mesenchymal cells that regulate hair follicle differentiation.

In vitro cultivation of epidermal cells, including hair follicle cells, has been difficult to achieve in the absence of unpurified biological components (such as serum or pituitary extract) or feeder cells to provide an adequate nutritional environment. Chemically defined media that permit in vitro proliferation, expansion and differentiation of murine and human epidermal cells are advantageous for developing physiologically accurate in vitro models of skin and hair growth, thus avoiding the need for animal-based research. Also, chemically defined media are preferred when growing epidermal cells for human therapeutic purposes, such as skin grafts. The use of chemically defined media reduces the risk of adverse effects presently caused by the undefined constituents in serum-supplemented cell culture media and feeder cell layers.

It is important to establish culture conditions which support the growth and differentiation of epidermal and follicular cells, particularly human cells and skin explants used to develop skin grafts. Presently, skin grafts do not contain hair follicles, sebaceous glands or eccrine glands. It is important to develop graft growth conditions which support the development of these epidermal substructures. Hair provides protection for the skin graft and is also important for aesthetic purposes. The incorporation of sebaceous glands into a skin graft will prevent the skin from being dry and flaky. Eccrine (sweat) glands are crucial for proper regulation of the body's response to changes in temperature.

### SUMMARY OF THE INVENTION

The invention provides for a chemically defined animal cell culture medium comprising (a) a synthetic basal medium; (b) calcium at a concentration of from about 1.2 mM to about 1.4 mM; (c) retinoid at a concentration of from about 0.01 mg/ml to about 1.0 mg/ml; (d) vitamin D at a concentration of from about 0.01 mg/ml to about 0.5 mg/ml; and (e) linoleic acid at a concentration of from about 0.01 mg/ml to about 1 mg/ml. In an embodiment, the synthetic basal medium comprises SPRD-111, SPRD-110, DMEM, Williams Medium E, Super Williams Medium (see Table 1), or any combination thereof. In one embodiment, the concentration of sodium is from about 7.6 mg/ml to about 7.5 mg/ml. In another embodiment, the concentration of potassium is from about 0.05 mg/ml to about 0.16 mg/ml. In yet another embodiment, the retinoid comprises retinyl acetate. In another aspect of the invention, the medium is suitable for culturing animal epidermal cells. In another embodiment, the culturing comprises cell expansion.

The invention also provides for a chemically defined animal cell culture medium composition comprising: (a) a synthetic basal medium; (b) insulin at a concentration of from about 2.5 mg/L to about 7.5 mg/L; (c) transferrin at a concentration of from about 5 mg/L to about 15 mg/L; (d) vitamin D₂ at a concentration of from about 0.5 mg/L to about 1.5 mg/L; (e) linoleic acid-BSA at a concentration of from about 0.05 mg/L to about 0.15 mg/L; (f) hydrocortisone at a concentration of from about 0.5 mg/L to about 1.5 mg/L; (g) epidermal growth factor (EGF) at a concentration of from about 5 μg/L to about 15 μg/L; (h) vitamin A at a concentration of from about 0.0575 mg/L to about 0.1725 mg/L; (i) phosphoethanolamine at a concentration of from about 2.8 mg/L to about 8.4 mg/L; and (j) ethanolamine at a concentration of from about 0.061 mg/L to about 0.183 mg/L. In one embodiment, the synthetic basal medium comprises SPRD-111, SPRD-110, DMEM, Williams Medium E, Super Williams Medium (see Table 1) or any combination thereof. In another embodiment, the medium further comprises delipidized bovine serum albumin (BSA) at a concentration of from about 0.5 g/L to about 1.7 g/L. In another embodiment, the medium comprises glutamine at a concentration of from about 1 mM to about 5 mM, penicillin at a concentration of from about 50 units/ml to about 150 units/ml, streptomycin at a concentration of from about 50 μg/ml to about 150 μg/ml, or any combination thereof.

The invention provides for a medium referred to as Morris 1 Medium, which comprises (a) Super Williams Medium (see Table 1); (b) insulin at a concentration of from about 2.5 mg/L to about 7.5 mg/L; (c) transferrin at a concentration of from about 5 mg/L to about 15 mg/L; (d) vitamin D₂ at a concentration of from about 0.5 mg/L to about 1.5 mg/L; (e) linoleic acid-BSA at a concentration of from about 0.05 mg/L to about 0.15 mg/L; (f) hydrocortisone at a concentration of from about 0.5 mg/L to about 1.5 mg/L; (g) epidermal growth factor (EGF) at a concentration of from about 5 μg/L to about 15 μg/L; (h) vitamin A at a concentration of from about 0.0575 mg/L to about 0.1725 mg/L; (i) phosphoethanolamine at a concentration of from about 2.8 mg/L to about 8.4 mg/L; and (j) ethanolamine at a concentration of from about 0.061 mg/L to about 0.183 mg/L. In another embodiment, the medium further comprises delipidized bovine serum albumin (BSA) at a concentration of from about 0.5 g/L to about 1.7 g/L. In another embodiment, the medium comprises glutamine at a concentration of from about 1 mM to about 5 mM, penicillin at a concentration of from about 50 units/ml to about 150 units/ml, streptomycin at a concentration of from about 50 μg/ml to about 150 μg/ml, or any combination thereof.

The invention further provides for a method of preparing a chemically defined animal cell culture medium, comprising mixing together the following: (a) a synthetic basal medium; (b) insulin at a concentration of from about 2.5 mg/L to about 7.5 mg/L; (c) transferrin at a concentration of from about 5 mg/L to about 15 mg/L; (d) vitamin D₂ at a concentration of from about 0.5 mg/L to about 1.5 mg/L; (e) linoleic acid-BSA at a concentration of from about 0.05 mg/L to about 0.15 mg/L; (f) hydrocortisone at a concentration of from about 0.5 mg/L to about 1.5 mg/L; (g) epidermal growth factor (EGF) at a concentration of from about 5 μg/L to about 15 μg/L; (h) vitamin A at a concentration of from about 0.0575 mg/L to about 0.1725 mg/L; (i) phosphoethanolamine at a concentration of from about 2.8 mg/L to about 8.4 mg/L; and (j) ethanolamine at a concentration of from about 0.061 mg/L to about 0.183 mg/L. In one embodiment, the synthetic basal medium comprises SPRD-111, SPRD-110, DMEM, Williams Medium E, Super Williams Medium (see Table 1) or any combination thereof. In another embodiment, the medium further comprises delipidized bovine serum albumin (BSA) at a concentration of from about 0.5 g/L to about 1.7 g/L. In another embodiment, the medium comprises glutamine at a concentration of from about 1 mM to about 5 mM, penicillin at a concentration of from about 50 units/ml to about 150 units/ml, streptomycin at a concentration of from about 50 μg/ml to about 150 μg/ml, or any combination thereof.

The invention also provides for a method for preparing a chemically defined animal cell culture medium, comprising mixing together the following: (a) a synthetic basal medium; (b) calcium at a concentration of from about 1.2 mM to about 1.4 mM; (c) retinoid at a concentration of from about 0.01 mg/ml to about 1.0 mg/ml; (d) vitamin D at a concentration of from about 0.01 mg/ml to about 0.5 mg/ml; and (e) linoleic acid at a concentration of from about 0.01 mg/ml to about 1 mg/ml. In one embodiment, the concentration of sodium is from about 7.6 mg/ml to about 7.5 mg/ml. In another embodiment, the concentration of potassium is from about 0.05 mg/ml to about 0.16 mg/ml. In yet another embodiment, the retinoid comprises retinyl acetate.

The invention also provides for a chemically defined medium comprising an inventive medium described above, wherein the medium comprises reduced concentrations of one or more factors that modulate cell growth. In one embodiment, the factor comprises epidermal growth factor, insulin, transferrin, retinoid, hydrocortisone, fibroblast growth factors, or any combination thereof.

In one embodiment of the invention, the medium is suitable for culturing mammalian cells. In one embodiment, the cultured cells are human cells. In another embodiment, the medium is suitable for culturing animal epidermal cells. In one embodiment, the culturing comprises cell differentiation. In another embodiment, the cells comprise hair follicle cells, keratinocytes, outer root sheath cells, hair matrix cells, hair follicle dermal papilla cells, skin fibroblasts, keratinocyte stem cells, follicular papillae, sheath cells, non-stem cell keratinocytes, bone marrow stem cells, melanocytes, sphere forming keratinocytes, mesenchymal cells or any combination thereof. In one embodiment, the cells are CD43 positive. In another embodiment, the medium is suitable for culturing sphere cells.

Provided for by this invention is a method for culturing whole hair follicles, the method comprising implanting a follicle into a culture contacting the implanted follicle with one of the inventive media.

The present invention also provides for a method for culturing hair follicle cells, the method comprising putting hair follicle cells in culture with one of the inventive media. In one embodiment, the hair follicle cells comprise cells of the follicular papillae, sheath cells, keratinocyte stem cells, bone marrow stem cells, melanocytes, sphere forming keratinocytes, mesenchymal cells or any combination thereof. In another embodiment, the culture does not comprise a feeder layer of cells. In an additional embodiment, the medium does not comprise serum.

In another aspect, the invention provides for a method for in vitro reconstruction of hair follicles, the method comprising (a) co-culturing epidermal keratinocytes with hair inductive mesenchymal cells, wherein the co-culturing is in the presence of a matrix; and (b) contacting the co-culture with one of the media of the invention. In a preferred embodiment, the hair inductive mesenchymal cells comprise keratinocyte stem cells, cells from the follicular papillae, sheath cells, or any combination thereof. In another embodiment, the co-culture does not comprise a feeder layer of cells. In an additional embodiment, the medium does not comprise serum.

The present invention also encompasses a method for identifying a whether a test compound is capable of modulating the activity of a hair follicle, the method comprising (a) contacting a hair follicle cultured according to a method of this invention with a test compound; (b) measuring the activity of the hair follicle in (a) compared to the activity of a hair follicle in the absence of the test compound, so as to identify whether the test compound is capable of modulating the activity of the hair follicle. In one embodiment, the activity of the hair follicle is measured as inhibition of hair growth, enhanced hair growth, or loss of hair from the follicle.

The present invention also provides for a method for culturing explants of mammalian skin, the method comprising contacting an explant of mammalian skin with one of the media of the invention. In a specific embodiment, the mammalian skin is human skin. In another embodiment, the culture does not comprise a feeder layer of cells. In yet another embodiment, the medium does not comprise serum. In a preferred embodiment, the explant is suitable for use as a skin graft.

In one aspect, the explant comprises functional hair follicles. In one embodiment, explant outgrowths comprise functional hair follicles. In another embodiment, the explant outgrowths comprise sebaceous glands. In an additional embodiment, the explant outgrowths comprise eccrine glands.

This invention provides for a method for identifying whether a test compound is capable of modulating hair growth, the method comprising, (a) contacting a test compound with a hair follicle cultured according to a method of this invention; and (b) assessing hair growth from the follicle in (a) compared to hair growth from a follicle in the absence of the test compound, so as to identify whether the test compound is capable of modulating hair growth.

This invention also provides for a method for identifying whether a test compound is capable of modulating the growth of skin, the method comprising (a) contacting a test compound with a skin explant cultured according to the methods of this invention; and (b) assessing the growth of the skin in (a) compared to the growth of skin in the absence of the test compound, so as to identify whether the test compound is capable of modulating the growth of skin.

One aspect of this invention provides for a method of culturing mammalian epithelial cells, comprising growing epithelial cells in vitro in the presence of one of the media provided for by the invention. In one embodiment, the cells comprise keratinocytes. In another embodiment, the culture does not comprise a feeder layer of cells. In another embodiment, the medium does not comprise unpurified or minimally-purified biological components, such as serum or pituitary extract.

Another aspect of the present invention provides for a method for growing hair follicles in the presence of one of the media provided for by the invention. In one embodiment, the cells comprise keratinocytes. In another embodiment, the culture does not comprise a feeder layer of cells. In another embodiment, the medium does not comprise unpurified serum or any other unpurified biological.

### BRIEF DESCRIPTION OF THE FIGURES

FIG. 1. Diameter of epithelial outgrowths from mouse skin explants (n=5) cultured for 3 weeks in SPRD-111 medium supplemented with various ratios of Na⁺/K⁺.

FIG. 2. Epithelial outgrowths of mouse skin explants cultured in SPRD-111 (Na⁺/K⁺ ratio=59.4), Williams Medium E (Na⁺/K⁺ ratio=15.67) or DMEM (Na⁺/K⁺ ratio=16.97).

FIGS. 3A-3B. Human keratinocytes were seeded at 5000/well and grown for 3 days on keratinocyte basal medium (KBM; Clonetics). Images are 100×.

FIGS. 4A-4B. Human keratinocytes were seeded at 5000/well and grown for 3 days on Williams Medium E containing 10% FBS and supplements. Images are 100×.

FIGS. 5A-5B. Human keratinocytes were seeded at 5000/well and grown for 3 days on Super Williams medium containing supplements (Morris 1 Medium). Images are 100×.

FIGS. 6A-6B. Human keratinocytes were seeded at 5000/well and grown for 3 days on Williams Medium E containing Super Williams supplements. Images are 100×.

FIGS. 7A-7B. Human keratinocytes were seeded at 10000/well and grown for 3 days on keratinocyte basal medium (KBM; Clonetics). Images are 100×.

FIGS. 8A-8B. Human keratinocytes were seeded at 10000/well and grown for 3 days on Williams Medium E containing 10% FBS and supplements. Images are 100×.

FIGS. 9A-9B. Human keratinocytes were seeded at 10000/well and grown for 3 days on Super Williams medium containing supplements (Morris 1 Medium). Images are 100×.

FIGS. 10A-10B. Human keratinocytes were seeded at 10000/well and grown for 3 days on Williams Medium E containing Super Williams supplements. Images are 100×.

FIGS. 11A-11B. Human keratinocytes were seeded at 5000/well and grown for 5 days on keratinocyte basal medium (KBM; Clonetics). Images are 100×.

FIGS. 12A-12B. Human keratinocytes were seeded at 5000/well and grown for 5 days on Williams Medium E containing 10% FBS and supplements. Images are 100×.

FIGS. 13A-13B. Human keratinocytes were seeded at 5000/well and grown for 5 days on Super Williams medium containing supplements (Morris 1 Medium). Images are 100×.

FIGS. 14A-14B. Human keratinocytes were seeded at 5000/well and grown for 5 days on Williams Medium E containing Super Williams supplements. Images are 100×.

FIGS. 15A-15B. Human keratinocytes were seeded at 10000/well and grown for 5 days on keratinocyte basal medium (KBM; Clonetics). Images are 100×.

FIGS. 16A-16B. Human keratinocytes were seeded at 10000/well and grown for 5 days on Williams Medium E containing 10% FBS and supplements. Images are 100×.

FIGS. 17A-17B. Human keratinocytes were seeded at 10000/well and grown for 5 days on Super Williams medium containing supplements (Morris 1 Medium). Images are 100×.

FIGS. 18A-18B. Human keratinocytes were seeded at 10000/well and grown for 5 days on Williams Medium E containing Super Williams supplements. Images are 100×.

FIG. 19. Human keratinocytes were seeded at 10000/well and grown for 17 days in Morris 1 medium. Images are 100×.

FIGS. 20A-20B. Primary human outer root sheath cells grown for 6 days (A) and 10 days (B) in Morris 1 medium. Images are 100×.

FIGS. 21A-21B. Human hair matrix cells grown for 2 days (A) and 15 days (B) in Morris 1 medium. Images are 100×.

FIGS. 22A-22B. Human hair follicle dermal papilla cells grown for 3 days (A) and 14 days (B) in Morris 1 medium. Images are 100×.

FIGS. 23A-23B. Human skin fibroblasts were seeded at 5000/well and grown for 4 days (A) and 13 days (B) in Morris 1 medium. Images are 100×.

FIGS. 24A-24B. Sphere cells from human scalp hair follicle outer root sheath cell culture grown for 14 days (A) and 21 days (B) in Morris 1 medium. Images are 100×.

FIGS. 25A-25B. Sphere cells from human scalp hair follicle cell culture grown for 11 days (A) and 20 days (B) in Morris 1 medium. Images are 100×.

FIGS. 26A-26B. Sphere cells from human abdomen hair follicle outer root sheath cell culture grown for 15 days (A) and 26 days (B) in Morris 1 medium. Images are 100×.

FIGS. 27A-27B. Sphere cells from CD34 positive human scalp hair follicle outer root sheath cell culture grown for 17 days (A) or 26 days (B) in Morris 1 medium. Images are 100×.

FIGS. 28A-28C. Sphere cells (P1) from human scalp hair follicle cell culture grown for 4 days (A), 9 days (B) and 10 day s (C) in Morris 1 medium. Images are 100×.

FIGS. 29A-29B. Attached sphere cells (P1) from human scalp hair follicle cell culture grown for 13 days in Morris 1 medium. Images are 100×.

FIGS. 30A-30B. Attached sphere cells (P2) from human scalp hair follicle cell culture grown for 3 days in Morris 1 medium. Images are 100×.

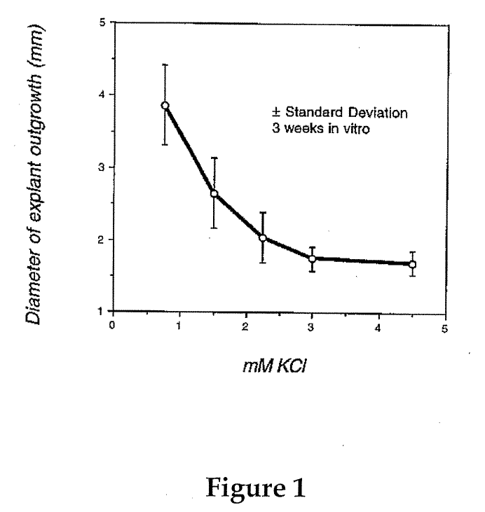

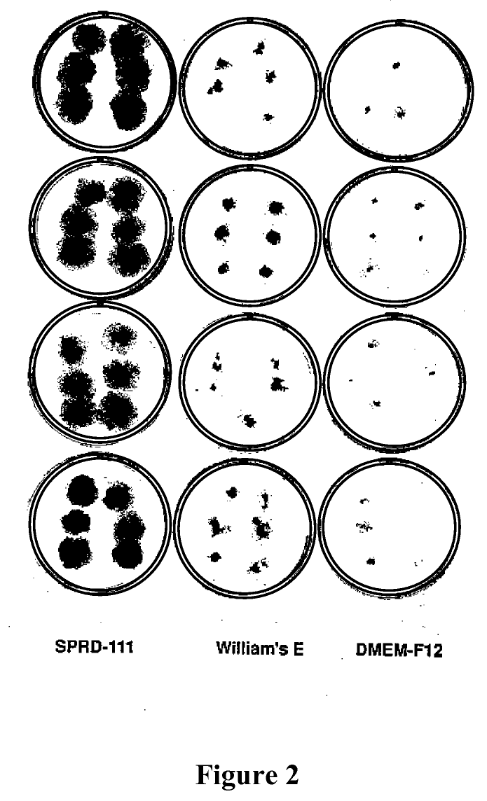

### DETAILED DESCRIPTION OF THE INVENTION

The patent and scientific literature referred to herein establishes knowledge that is available to those skilled in the art. The issued patents, applications, and other publications that are cited herein are hereby incorporated by reference to the same extent as if each was specifically and individually indicated to be incorporated by reference.

The medium provided for by the invention has a high ratio of sodium to potassium as does SPRD-111 but has a simpler composition and a longer shelf life. This ratio is in the range of from about 57.5 to about 27.9. In this medium, the calcium concentration is in the range from about 1.2 millimolar to about 1.4 millimolar. The medium can be used as a chemically defined medium for cultivating mouse and human epidermal keratinocytes. The medium may be useful for cultivating human keratinocytes for use in methods of grafting the cultured keratinocytes onto burn patients and for gene therapy or bioengineering applications. One problem that the medium solves is that mouse cells do not grow well in the media currently used. This chemically defined medium of the present invention solves this problem because mouse cells grow better in the media of the invention then previous media. This is important because there is a need in the biotechnology and pharmaceutical industry to stop using animal models in research and development. A media that permits robust growth of mouse cells, and especially keratinocytes and epidermal cells of mouse and human (and other mammals) will take the place of animal models now being used. The need for non-animal cell culture methods for testing products, drugs and other items is fulfilled by the present invention. Furthermore, a medium that allows growth of human epidermal cells without a feeder layer (such as 3T3 cells) is needed for cultures supplying graft material for burn patients.

The present invention provides for chemically defined culture media for establishing and cultivating animal cells, preferably epidermal cells. U.S. Pat. Nos. 5,126,261 and 5,266,479 disclose other formulations of chemically defined culture media useful for culturing epidermal cells. The media of the present invention are improved over previous formulations because they are chemically less complex, therefore easier to make, and they have a longer shelf-life. In addition, the media of the present invention include a specific sodium/potassium ratio and growth supplements. Although the previous media formulations supported the outgrowth of epidermal cells from cultured explants of mouse epidermis, and the proliferation and differentiation of adult human and murine epidermal keratinocytes, the previous formulations could not effectively support the expansion of primary cultures of murine epidermal cells (See Morris et al., In Vitro Cell Dev Biol 27A:886-895 (1991)). An improvement in the sodium to potassium ratios encompassed by the present invention has resulted in the successful expansion of adult murine epidermal cells.

The present invention provides for a chemically defined animal cell culture media suitable for expansion of epidermal cells and follicular cells. Expansion media are rich in factors that support growth and proliferation of the cultured cells. The invention also provides for chemically defined cell culture media suitable for supporting differentiation of epidermal cells and follicular cells. Differentiation media are pared-down, minimal versions of the expansion media, and comprise reduced concentrations of factors that support cell growth. The differentiation media slow the growth rate of the cultured cells, thus giving the cells an opportunity to differentiate.

The inventive media are particularly useful for in vitro cultivation of epidermal hair follicle cells, including follicular stem cell keratinocytes and non-stem cell keratinocytes, follicular sheath cells, and cells of the follicular papillae. The invention provides for culture conditions for growing and expanding murine and human hair follicle cells, and for the co-culture of one or more follicular cell types for the in vitro reconstruction of hair follicles.

The development of a chemically defined culture medium is preferred over commonly-used media supplemented with unpurified or minimally purified biological components, such as serum or pituitary extract. The addition of biological components to culture medium is required to supply the cultured cells with an optimal nutritional environment, but the biological components also expose the cells to a large number of undefined compounds which may inadvertently and undesirably affect cell biology. The undefined composition of media supplemented with biologicals precludes the use of these biological products in humans. Additionally, fetal bovine serum is the serum of choice for this purpose, leading to risks of outbreaks of Creutzfeldt-Jakob Disease (CJD), the human equivalent of bovine spongiform encephalopathy (BSE) or “mad-cow disease.” In contrast, chemically defined media do not utilize serum, or utilize only highly purified biological components, and the media constituents are known and present in defined quantities.

Serum-free methods have been established for cultivation and expansion of human keratinocytes. Generally, this method requires that the keratinocytes are grown on a layer of feeder cells, typically mouse 3T3 fibroblasts (See Wu and Morris, Methods Mol Biol 289:79-86 (2005)). However, this method is also undesirable in that keratinocytes grown in this manner cannot be used therapeutically in humans. The mouse feeder cells can induce genetic changes in the human keratinocytes. For example, human keratinocytes cultured on a layer of mouse 3T3 feeder cells were found to express murine-specific antigens, raising the possibility that transplanted grafts grown on mouse feeder cells could trigger the human host immune response, ultimately resulting in loss of the graft (Cairns et al., Journal of Trauma-Injury Infection & Critical Care. 39:75-80 (1995)). To prevent growth of feeder layer cells, the cells are subjected to irradiation or alkylating agents (such as mitomycin) to induce DNA cross-linking. These treatments can induce mutations in the feeder cells that may be introduced into the proliferating cultured cells. Oncogene transmission is also a risk when culturing human cells on mouse feeder cells. The mouse cells may introduce an oncogene into the human cells, resulting in transformation of the human cells into a tumor-like phenotype. The possibility of the introduction of genetic changes in cultures of human keratinocytes underscores the disadvantages of using feeder layers. A recent study aimed at circumventing these problems has demonstrated growth and expansion of human keratinocytes under serum-free conditions on a feeder layer of non-irradiated human fibroblasts (Sun et al., Wound Repair Regen 12:626-634 (2004)). However, this method does not provide completely defined culture conditions because not every factor released by the fibroblast layer has been defined.

The chemically defined cell culture media of the present invention surpass the need for serum and feeder layers and are thus desirable for establishing and cultivating human epidermal cells for therapeutic purposes, such as skin grafts.

Media Ingredients and Preparation

The present invention provides for a chemically defined animal cell culture medium comprising (a) a synthetic basal medium; (b) calcium at a concentration of from about 1.2 mM to about 1.4 mM; (c) a ratio of sodium to potassium from about 57.5 to about 27.9; (d) retinoid at a concentration of from about 0.01 mg/ml to about 1.0 mg/ml; (e) vitamin D at a concentration of from about 0.01 mg/ml to about 0.5 mg/ml; and (f) linoleic acid at a concentration of from about 0.01 mg/ml to about 1 mg/ml. In one embodiment, the synthetic basal medium comprises medium comprising SPRD-111, DMEM, Williams Medium E, or Super Williams Medium (see Table 1). In one embodiment, the concentration of sodium is from about 7.6 mg/mil to about 7.5 mg/ml. In another embodiment, the concentration of potassium is from about 0.05 mg/ml to about 0.16 mg/ml. In yet another embodiment, the retinoid comprises retinyl acetate. In another aspect of the invention, the medium is suitable for culturing animal epidermal cells. In another embodiment, the culturing comprises cell expansion. In another embodiment, the epidermal cells comprise keratinocyte stem cells, follicular papillae, sheath cells, non-stem cell keratinocytes, or any combination thereof.

The invention also provides for a chemically defined medium comprising the medium described above, wherein the medium comprises reduced concentrations of one or more factors that modulate cell growth. In one embodiment, the factor comprises endothelial growth factor, insulin, transferrin, retinoid, or any combination thereof. In another embodiment, the medium is suitable for culturing animal epidermal cells. In another embodiment, the culturing comprises cell differentiation. In one embodiment, the epidermal cells comprise keratinocyte stem cells, follicular papillae, sheath cells, non-stem cell keratinocytes, or any combination thereof.

The invention also provides for a method for preparing a chemically defined animal cell culture medium, comprising mixing together the following (a) a synthetic basal medium; (b) calcium at a concentration of from about 1.2 mM to about 1.4 mM; (c) a ratio of sodium to potassium from about 57.5 to about 27.9; (d) retinoid at a concentration of from about 0.01 mg/ml to about 1.0 mg/ml; (e) vitamin D at a concentration of from about 0.01 mg/ml to about 0.5 mg/ml; and (f) linoleic acid at a concentration of from about 0.01 mg/ml to about 1 mg/ml. In one embodiment, the synthetic basal medium comprises comprising SPRD-111, DMEM, Williams Medium E, or Super Williams Medium (see Table 1). In one embodiment, the concentration of sodium is from about 7.6 mg/ml to about 7.5 mg/ml. In another embodiment, the concentration of potassium is from about 0.05 mg/ml to about 0.16 mg/ml.

SPRD-111 can be prepared as described in U.S. Pat. Nos. 5,126,261 and 5,266,479 (also see Morris et al., In Vitro Cell Dev Biol 27A:886-895 (1991)). Commercially available media can be used as the basal medium in the present invention. MCDB-151, the base medium for SPRD-111, is available from commercial vendors, including Irvine Scientific (cat. no. 9061). DMEM and Williams Medium E are available from vendors such as GIBCO or BioWhittaker. Super Williams can be prepared as shown in Table 1.

In Vitro Culture Methods

There are currently no adequate in vitro models for studying the modulation of hair follicle development or function. Current technology involves grafting component cells onto athymic mice (Scandurro et al., J Invest Dermatol 105:177-183 (1995); Miyashita et al., Exp Dermatol 13:491-498 (2004); Lichti et al, J Invest Dermatol 101 (1 Supp):124S-129S (1993); Jahoda et al., Exp Dermatol 10:229-237 (2001)), embedding individual rat vibrissae in vitro (Reynolds & Jahoda, J Dermatol Sci 7 Suppl:S84-97 (1994); Philpott & Kealey, J Invest Dermatol 115:1152-1155 (2000)) or co-culture of human follicular components.

Provided for by this invention is a method for culturing whole hair follicles, the method comprising implanting a hair follicle from into a culture and contacting the implanted follicle with one of the inventive media.

Methods for isolating whole hair follicles are described in Philpott et al., J Cell Sci 97(Pt. 3):463-471 (1990); Philpott & Kealey, J Invest Dermatol 115:1152-1155 (2000); Imai et al., Arch Drmatol Res 284:466-471 (1993); Moll, Arch Dermatol Res 288:604-610 (1996); Philpott et al., Dermatol Clin 14:595-607 (1996).

The present invention also provides for a method for culturing hair follicle cells, the method comprising contacting hair follicle cells with one of the inventive media. In a preferred embodiment, the hair follicle cells comprise cells of the follicular papillae, sheath cells, keratinocyte stem cells or any combination thereof. In another embodiment, the culturing is in the absence of a feeder layer of cells. In an additional embodiment, the culturing is in the absence of serum.

Methods for establishing, cultivating and expanding cultures of mammalian dermal papilla cells are described in U.S. Pat. No. 5,851,831 (see also Inamatsu et al., J Invest Dermatol 111:767-775 (1998) and Magerl et al., Exp Dermatol 1:381-385 (2002)). The described methods require the papilla cells to be cultured in the presence of epidermal feeder cells or in medium conditioned by the epidermal feeder cells. The use of feeder cells or conditioned media is undesirable due to the undefined factors produced by the feeder cells and the unknown effects of the factors on the papilla cells. Populations of follicular papilla cells are distinguished from other hair follicle cells by the expression of protease nexin 1 (Jensen et al., J Invest Dermatol 114:917-922 (2000)). U.S. Pat. Nos. 6,548,058 and 6,730,513 describe isolation and culture of follicular sheath cells using growth-arrested human fibroblasts as a feeder layer (See also Limat and Hunziker, Cells Tissues Organs 172:79-85 (2002) and Limat et al., Arch Dermatol Res 285:205-210 (1993)). Outer root sheath cells are marked by expression of nestin (Li et al., Proc Natl Acad Sci USA 100:9958-9961 (2003)) and keratin 14 (Gho et al., Br J Dermatol 150:860-868 (2004); Pena et al., EMBO J. 18:3596-3603 (1999)).

Methods for isolation and culture of human and murine non-stem cell epidermal keratinocytes are described in detail in Fischer et al., Mol Carcinog 7:228-237 (1993) and Cameron et al., Toxicol In Vitro 6:109-118 (1992). Non-stem cell keratinocytes are characterized by expression of keratin 10 (Webb et al., Differentiation 72:387-395 (2004)).

U.S. Pat. No. 5,556,783 describes methods for identifying and isolating follicular keratinocyte stem cells and cultivating the stem cells in the presence of a fibroblast feeder layer. Epidermal keratinocyte stem cells have been maintained in long-term culture on a fibroblast feeder layer (Papini et al., Stem Cells 21:481-494 (2003)). A population of keratinocyte stem cells can be identified by the expression of specific markers, including, but not limited to b1-integrin, keratin 15, keratin 19, CD71 (transferrin receptor), transcription factor P63 and CD34 (For review see Ma et al., Ann Acad Med Singapore 33:784-788 (2004)). Alpha-6 integrin, a marker of proliferative (basal) keratinocytes, can be used in conjunction with other markers such as CD34, keratin 15, and CD71 to enrich for hair follicle stem cells. (Tani et al., Proc Natl Acad Sci USA 97:10960-10965 (2000); Li et al., Proc Natl Acad Sci USA 95:3902-3907 (1998); Webb et al., Differentiation 72:387-395 (2004)).

Detection of cell-specific biomarkers can be accomplished by methods known in the art. For example, proteins can be detected by immunostaining techniques utilizing detectable antibodies. If protein levels are too low to be identified by immunostaining, PCR can be used to detect expression levels of the corresponding genes.

In another aspect, the invention provides for a method for in vitro reconstruction of hair follicles, the method comprising (a) co-culturing epidermal keratinocytes with hair inductive mesenchymal cells, wherein the co-culturing is in the presence of a matrix; and (b) contacting the co-culture with one of the media of the invention. In a preferred embodiment, the hair inductive mesenchymal cells comprise keratinocyte stem cells, cells from the follicular papillae, sheath cells, or any combination thereof. In another embodiment, the culturing is in the absence of a feeder layer of cells. In an additional embodiment, the culturing is in the absence of serum.

Organotypic cultures of hair follicle cells are attained by growing populations of follicular cell types in combination on a three-dimensional cell culture matrix. Non-limiting examples of matrices include collagen, fibronectin, basement membrane Matrigel™ (BD Biosciences), and Vitrogen® 100 fibrillar collagen films. Toward this end, three-dimensional follicle-like structures have been observed to form spontaneously upon co-culture of mouse epidermal keratinocytes and hair inductive mouse mesenchymal cells.

One study of hair follicle reconstruction in vitro was based on the combined culture of four follicular cell types: outer root sheath cells, dermal papilla, dermal sheath, and germinative epidermal cells (Reynolds and Jahoda, J Dermatol Sci 7 Suppl:S84-S97 (1994)). Unlike the present invention, which provides for completely defined culture conditions, Reynolds and Jahoda used natural rat vibrissae sacks as containers to facilitate the recombination of the cultured component cells.

The present invention also provides for a method for culturing explants of mammalian skin, the method comprising (a) establishing the explant on a culture matrix; and (b) contacting the explant with one of the media of the invention. In a specific embodiment, the mammalian skin is human skin. In another embodiment, the culturing is in the absence of a feeder layer of cells. In yet another embodiment, the culturing is in the absence of serum. In a preferred embodiment, the explant is suitable for use as a skin graft.

In one aspect, the explant comprises functional hair follicles. In one embodiment, explant outgrowths comprise functional hair follicles. In another embodiment, the explant outgrowths comprise sebaceous glands. In an additional embodiment, the explant outgrowths comprise eccrine glands.

Explant cultures of mouse skin comprising hair follicles were grown in the presence of the medium provided for by this invention. When the cultured explants of mouse skin were grown over several weeks, hair lengthening and unexpected retention of follicle morphology were observed. It was found that hair follicles which were functional in intact mouse skin remained functional in the explant continued to function (i.e., hair continued to grow) when the explant was maintained in the expansion medium. Cultivation of the mouse skin explants on a Matrigel substratum led to a variety of tubular and follicle-like structures in the explant outgrowth. Methods for establishing a culture of a mouse skin explant are described in detail in U.S. Pat. Nos. 5,126,261 and 5,266,479 (also see Morris et al., In Vitro Cell Dev Biol 27A:886-895 (1991)).

The media of the present invention, including the expansion medium and the differentiation medium, are particularly useful for in vitro reconstruction of hair follicles and growth of hear-bearing skin explants. The formulation of the expansion medium allows for the rapid growth of cells in culture. When cultured in the expansion medium, hair follicle cells will proliferate rapidly. To facilitate the in vitro reconstruction of a hair follicle, it is preferable that cell growth is slowed when the hair follicle cell types are co-cultured, thus giving the cells an opportunity to properly recombine into a functional hair follicle. For the growth of hair-bearing skin explants in culture, it is desirable to reduce the growth rate of the explant outgrowths to ensure that the follicular cells are given adequate opportunity to differentiate and form functional hair follicles in the outgrowths. The formulation of the differentiation medium slows the growth rate of the epidermal cells, while still providing the correct nutritional environment for proper differentiation of the cells.

One aspect of this invention provides for a method of culturing mammalian epithelial cells, comprising growing epithelial cells in vitro in the presence of one of the media provided for by the invention. In one embodiment, the cells comprise keratinocytes. In another embodiment, the growing is in the absence of a feeder layer of cells. In another embodiment, the growing is in the absence of serum.

Another aspect of the present invention provides for a method for growing hair follicles in the presence a medium provided for by the invention. In one embodiment, the growing is in the absence of a feeder layer of cells. In another embodiment, the growing is in the absence of serum.

This invention provides for epidermal skin substitutes which may offer a novel therapeutic alternative to autologous skin grafts, currently widely used in wound repair, skin reconstruction after surgery, tissue replacement in burn victims, treatment of chronic ulcers, and hair restoration.

Compound Screening Assays

The present invention also encompasses a method for identifying a whether a test compound is capable of modulating the activity of a hair follicle, the method comprising (a) contacting a test compound with a hair follicle cultured according to a method of this invention; (b) measuring the activity of the hair follicle in (a) compared to the activity of a hair follicle in the absence of the test compound, so as to identify whether the test compound is capable of modulating the activity of the hair follicle. In one embodiment, the activity of the hair follicle is measured as inhibition of hair growth, enhanced hair growth, or loss of hair from the follicle.

This invention provides for a method for identifying whether a test compound is capable of modulating hair growth, the method comprising, (a) contacting a test compound with a hair follicle cultured according to a method of this invention; and (b) assessing hair growth from the follicle in (a) compared to hair growth from a follicle in the absence of the test compound, so as to identify whether the test compound is capable of modulating hair growth.

This invention also provides for a method for identifying whether a test compound is capable of modulating the growth of skin, the method comprising (a) contacting a test compound with a skin explant cultured according to the methods of this invention; and (b) assessing the growth of the skin in (a) compared to the activity of skin in the absence of the test compound, so as to identify whether the test compound is capable of modulating the growth of skin.

In vitro skin models, in vitro hair models and screening assays provided by this invention offer alternatives for current animal-based research, especially research conducted on carcinogens, tumor promoters, irritants, toxins and cosmetics.

The following examples illustrate the present invention, and are set forth to aid in the understanding of the invention, and should not be construed to limit in any way the scope of the invention as defined in the claims which follow thereafter.

### EXAMPLES

### Example 1

### In Vitro Isolation and Expansion of Cell Populations from Hair-Bearing Human Skin

Alpha-6 integrin+/CD34+ keratinocyte stem cells from the hair follicle bulge; mesenchymal cells from the follicular papillae and connective tissue sheaths; and undifferentiated mesenchymal sphere-forming stem cells are isolated by microdissection and by sphere-formation, respectively, according to published procedures. The expanded cell culture populations are compared with the freshly isolated cells using several physical and functional determinants of each population, such as expression of cytokeratins, nestin, and other markers, and by in vitro assay for colony formation. The follicle-forming potential of recombined primary and cultured cells is tested on de-epidermized dermis and by in vitro co-culture. Culture conditions can be optimized to achieve long-term maintenance of stem cell and follicle-forming properties of each follicular component.

### Example 2

### Assessment of the Hair-Growth Potential of Human Skin Explant Cultures

Explants of hair-bearing human skin (obtained from discarded specimens of hair-bearing human skin) are established on Transwell inserts and cultured at the air-liquid interface. Hair length is measured weekly using a calibrated dissecting microscope. Additionally, at bi-weekly intervals, several explants are removed and processed for light microscopy to assess the integrity of the follicles. Explants of human skin can also be established on Matrigel and the keratinocyte and fibroblast outgrowth monitored for formation of tubes and follicle-like structures. At bi-weekly intervals, several explants and their outgrowths are removed for histology. Culture conditions can be optimized such that the hair-bearing explants of human skin will demonstrate hair growth in culture.

### Example 3

### Concentration of Sodium, Potassium, Calcium and Magnesium in Basal Media

SPRD-111

SPRD-111 contains the following concentrations of compounds containing sodium, potassium, calcium and magnesium: sodium acetate (CH₃CO₂Na.3H₂O), 84.5 mg/ml; sodium pyruvate (C₃H₃NaO₃), 11.5 mg/ml; sodium phosphate (Na₂HPO₄), 92.0 mg/ml; sodium chloride (NaCl), 2990.70 mg/ml; sodium bicarbonate (NaHCO₃), 321.96 mg/ml; sodium sulfate (NaSO₄), 1.14 mg/ml; potassium chloride (KCl), 58.94 mg/ml; magnesium chloride (MgCl₂-6H₂O), 29.16 mg/ml; calcium chloride (CaCl₂), 48.41 mg/ml. The sodium to potassium ratio (Na⁺/K⁺) ratio of SPRD-111 is 59.4.

Williams Medium E

Williams Medium E contains the following amounts of compounds containing sodium, potassium, calcium and magnesium: sodium pyruvate (C₃H₃NaO₃), 5.23 mg/ml; sodium chloride (NaCl), 2676.25 mg/ml; sodium bicarbonate (NaHCO₃), 602.31 mg/ml; sodium phosphate (NaH₂PO₄.H₂O), 20.13 mg/ml; potassium chloride (KCl), 210.81 mg/ml; magnesium sulfate (MgSO₄), 19.72 mg/ml; magnesium chloride (MgCl₂.6H₂O), 14.58 mg/ml; calcium chloride (CaCl₂), 72.22 mg/ml. The sodium to potassium ratio (Na⁺/K⁺) ratio of Williams Medium E is 15.67.

DMEM

DMEM (Dulbecco's Modified Eagle Medium) contains the following concentrations of compounds containing sodium, potassium, calcium and magnesium: sodium pyruvate (C₃H₃NaO₃), 11.5 mg/ml; sodium phosphate (Na₂HPO₄), 35.94 mg/ml; sodium chloride (NaCl), 2518.82 mg/ml; sodium bicarbonate (NaHCO₃), 1013.10 mg/ml; potassium chloride (KCl), 210.81 mg/ml; magnesium sulfate (MgSO₄), 19.72 mg/ml; calcium chloride (CaCl₂), 72.22 mg/ml. The sodium to potassium ratio (Na⁺/K⁺) ratio of DMEM is 16.97.

### Example 4

### Preparation of Super William's Medium (High Calcium)

The components of Super William's Medium are listed in Table 1. The concentration of each component is listed in milligrams based on a final medium volume of 1

TABLE 1
Super William's Medium (high Ca)

| INGREDIENT | 1x (mg/ml) |
|---|---|
| stock 1 | |
| Arginine * HCl | 60.5 |
| Histidine * HCl * H20 | 20.3 |
| Iso-leucine | 50 |
| Leucine | 75 |
| Lysine * HCl | 87.5 |
| Methionine | 15 |
| Phenylalanine | 25 |
| Threonine | 40 |
| Tryptophan | 10 |
| Tyrosine | 35 |
| Valine | 50 |
| Choline Chloride | 13.96 |
| Serine | 10 |
| stock 2 | |
| Biotin | 0.5 |
| Ca D-pantothenate | 1 |
| Niacinamide | 1 |
| Pyridoxine * HCl | 1 |
| Thiamine * HCl | 0.337 |
| KCl | 111.83 |
| stock 3 | |
| NaH2PO4 | 140 |
| Folic acid | 0.794 |
| stock 4 | |
| MgSO4 | 97.67 |
| FeSO4 * 7H20 | 0.417 |
| CaCl2 | 200 |
| stock 5 | |
| phenol red | 10 |
| stock 6 | |
| Glutathione | 0.05 |
| Na pyruvate | 55 |
| riboflavin | 0.1 |
| stock 7 | |
| Cysteine * HCl * H20 | 58 |
| Cystine * 2HCl | 26.1 |
| stock 8 | |
| Asparagine | 20 |
| Proline | 30 |
| Vit B12 | 0.68 |
| stock 9 | |
| Alanine | 90 |
| Aspartic Acid | 30 |
| Glutamic Acid | 50 |
| Glycine | 50 |
| stock 10 | |
| L inositol | 18.02 |
| Adenine * HCl | 30.89 |
| Lipoic Acid | 0.206 |
| Thymidine | 0.727 |
| CuSO4 * 5H2O | 0.0001 |
| ZnSO4 * 7H2O | 0.0002 |
| stock 11 | |
| Ascorbic Acid | 2 |
| Menadione | 0.01 |
| stock 12 | |
| Glucose | 2000 |
| NaCl | 6995.95 |
| NaHC03 | 2200 |

liter.
The pH is adjusted to 7.2-7.4. The following stocks are then added: 1 μl 0.03 mg/μl methyl linoleate, 10 μl Vitamin D2 stock, and 7.8 μl 1 mg/ml Tocophenol, 100 μl Vitamin A stock. The solution is then brought to a final volume of one liter.

### Example 5

### Preparation of Supplements for Addition to Basal Medium

One or more of the following supplements may be added to a basal medium before using. When the addition of water is indicated, 2× distilled reverse osmosis (RO) water is recommended.

Delipidized Bovine Serum Albumin

Delipidized bovine serum albumin (BSA) was obtained from Collaborative Research (#40331). Stock solutions of BSA are sterile filtered, for example, with a 0.2 μm filter, before addition to the medium. Stock solutions may be aliquotted and stored at −20° C. 0.565 g of BSA are added to one 500 ml bottle of basal medium resulting in a final BSA concentration of 1.13 g/L. The final concentration of BSA in the medium may be from about 0.5 g/L to about 1.7 g/L.

Insulin

Insulin was obtained from Collaborative Research (#40310). Stock solutions of insulin are sterile filtered, for example, with a 0.2 μm filter, before addition to the medium. Stock solutions may be aliquotted and stored at −20° C. 2.5 mg insulin are added to one 500 ml bottle of basal medium (final concentration of 5 mg/L insulin). The final concentration of insulin in the medium may be from about 2.5 mg/L to about 7.5 mg/L.

Transferrin

Transferrin was obtained from Sigma (#T1147). Stock solutions of transferrin are sterile filtered, for example, with a 0.2 μm filter, before addition to the medium. Stock solutions may be aliquotted and stored at −20° C. 5 mg transferrin are added to one 500 ml bottle of basal medium (final concentration 10 mg/L). The final concentration of transferrin in the medium may be from about 5 mg/L to about 15 mg/L.

Vitamin D2 (Ergocalciferol)

Vitamin D₂ was obtained from Sigma. Stock solutions of vitamin D₂ are prepared in absolute ethanol and are sterile filtered, for example, with a 0.2 μm filter, before addition to the medium. Stock solutions may be aliquotted and stored at −20° C. 0.5 mg vitamin D₂ are added to one 500 ml bottle of basal medium (final concentration 1 mg/L). The final concentration of vitamin D₂ in the medium may be from about 0.5 mg/L to about 1.5 mg/L.

Linoleic Acid-BSA

Linoleic acid-BSA was obtained from Collaborative Research (#40227). Stock solutions of linoleic acid-BSA are sterile filtered, for example, with a 0.2 μm filter, before addition to the medium. Stock solutions may be aliquotted and stored at −20° C. 0.05 mg linoleic acid-BSA are added to one 500 ml bottle of basal medium (final concentration 0.1 mg/L). The final concentration of linoleic acid-BSA in the medium may be from about 0.05 mg/L to about 0.15 mg/L.

Hydrocortisone

Hydrocortisone was obtained from Collaborative Research (#40203). Hydrocortisone solutions are prepared in absolute ethanol. Stock solutions of hydrocortisone are sterile filtered, for example, with a 0.2 μm filter, before addition to the medium. Stock solutions may be aliquotted and stored at −20° C. 0.5 mg hydrocortisone are added to one 500 ml bottle of basal medium (final concentration 1 mg/L). The final concentration of hydrocortisone in the medium may be from about 0.5 mg/L to about 1.5 mg/L.

Epidermal Growth Factor (EGF)

EGF was obtained from Collaborative Research (#40001). EGF solutions are prepared in sterile water. 5 μg of EGF are added to one 500 ml bottle of basal medium (final concentration 10 μg/L). The final concentration of EGF in the medium may be from about 5 μg/L to about 15 μg/L.

Glutamine

Glutamine was obtained as a sterile 200 mM solution from Whittaker M.A. Bioproducts (#17-605B). Thawed glutamine must be warmed to dissolve precipitate. Stocks of glutamine can be stored at −20° C. 5 ml of glutamine are added to one 500 ml bottle of basal medium (final concentration 2 mM). The final concentration of glutamine in the medium may be from about 1 mM to about 5 mM.

Phosphoethanolamine

Phosphoethanolamine was obtained from Sigma (#P0503). The powder is stored dessicated in a freezer. Stock solutions of hydrocortisone are prepared in PBS and sterile filtered, for example, with a 0.2 μm filter, before addition to the medium. Stock solutions may be aliquotted and stored at −20° C. 2.8 mg phosphoethanolamine are added to one 500 ml bottle of basal medium (final concentration 5.6 mg/L). The final concentration of phosphoethanolamine in the medium may be from about 2.8 mg/L to about 8.4 mg/L.

Ethanolamine

Ethanolamine was obtained from Sigma (#E0135). Stocks of ethanolamine are prepared in water and sterile filtered, for example, with a 0.2 μm filter, before addition to the medium. Ethanolamine is prepared fresh for each use. Concentrated stocks of ethanolamine are diluted in PBS to the desired working concentration. 0.0611 mg ethanolamine are added to one 500 ml bottle of basal medium (final concentration 0.122 mg/L). The final concentration of ethanolamine in the medium may be from about 0.061 mg/L to about 0.183 mg/L.

Penicillin-Streptomycin

Penicillin-streptomycin was obtained from GIBCO (#15140-122) or Whittaker M.A. Bioproducts (#17-602A). Stock solutions may be aliquotted and stored at −20° C. 50,000 units of penicillin are added to one 500 ml bottle of basal medium (final concentration 100 units/ml). The final concentration of penicillin may be from about 50 units/ml to about 150 units/ml. 50,000 μg of streptomycin are added to one bottle of basal medium (final concentration of 100 μg/ml). The final concentration of streptomycin may be from about 50 μg/ml to about 150 μg/ml.

Vitamin A (Retinyl Acetate)

Vitamin A was obtained from GIBCO (#33000-019). Stocks of vitamin A are prepared in absolute ethanol and set aside in the dark and allowed to go into solution (light sensitive). Solutions of vitamin A are sterile filtered, for example, with a 0.2 μm filter, before addition to the medium. Aloquots of vitamin A stock solutions are stored at −20° C. 0.0575 mg vitamin A are added to one 500 ml bottle of basal medium (final concentration 0.115 mg/L). The final concentration of vitamin A in the medium may be from about 0.0575 mg/L to about 0.1725 mg/L.

### Example 6

### Growth of Human Keratinocyte Cultures on Off-the-Shelf Media Compared to the Chemically-Defined Media Provided by the Invention

Growth of human keratinocyte cultures grown on a commercially available off-the-shelf keratinocyte medium (keratinocyte basal media (KBM) from Clonetics) was compared to growth of human keratinocyte cultures grown on supplemented off-the-shelf media (Williams Medium E) provided by the invention.

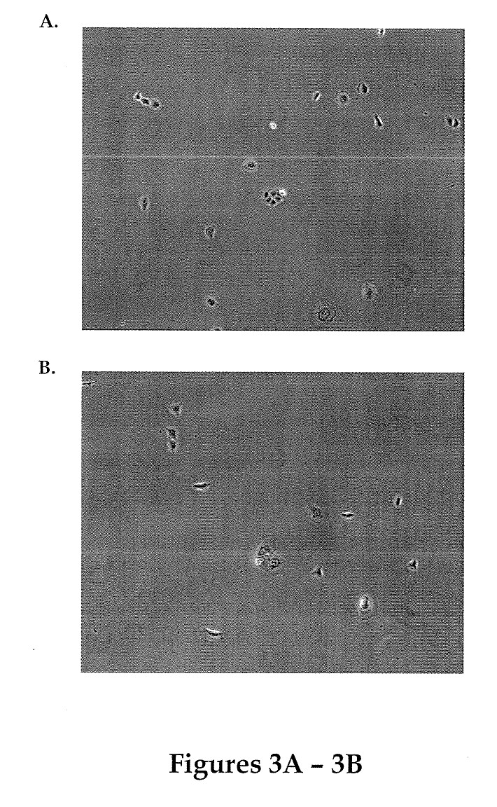

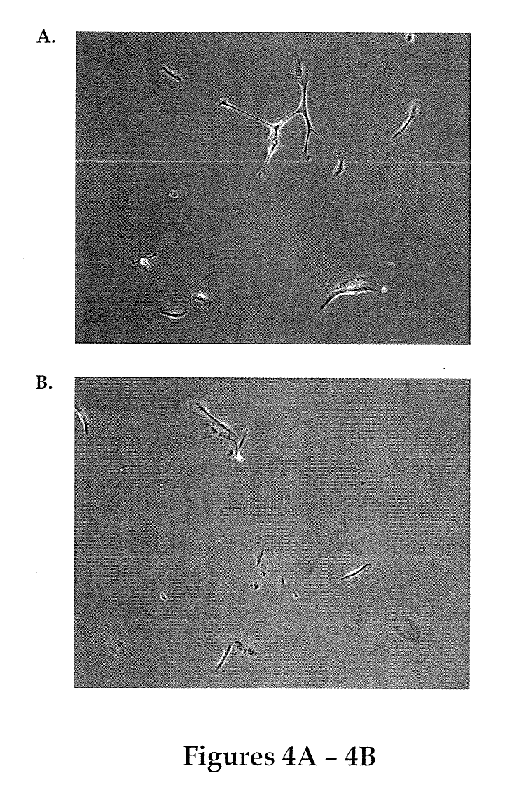

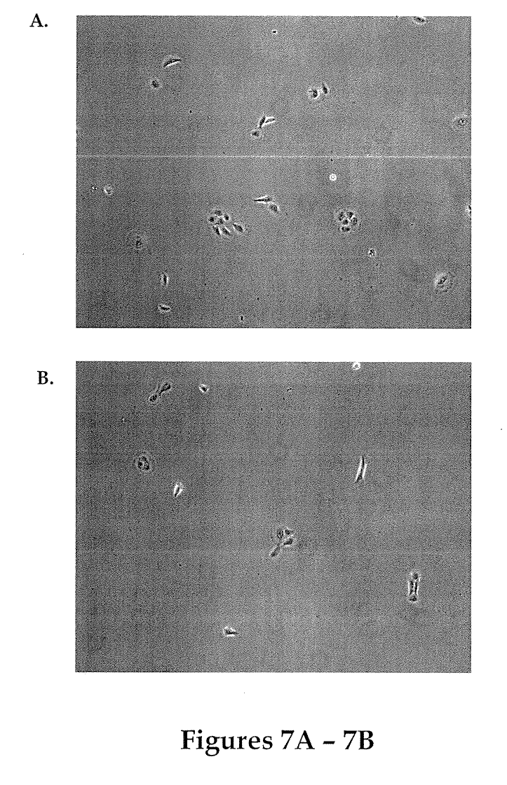

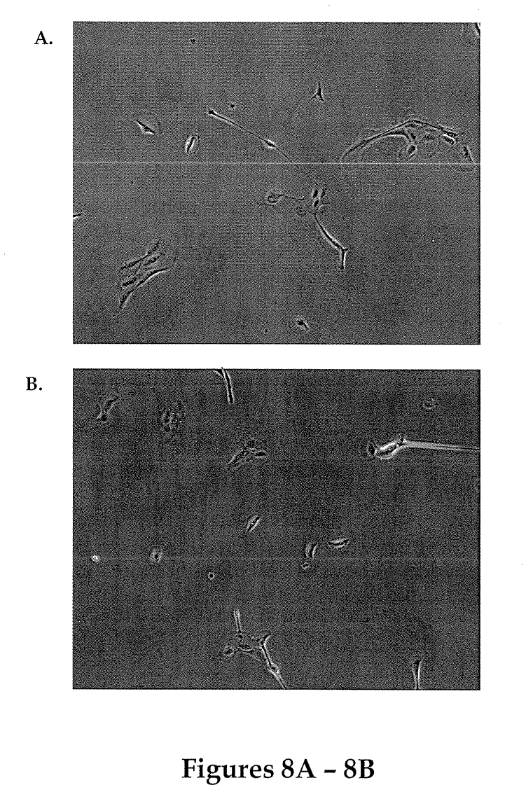

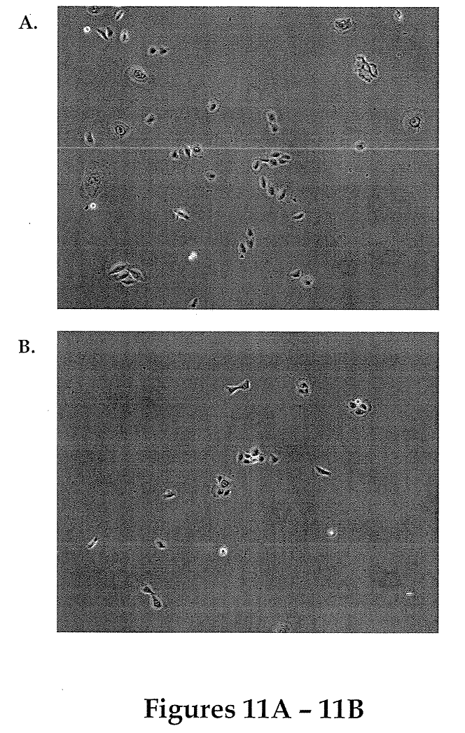

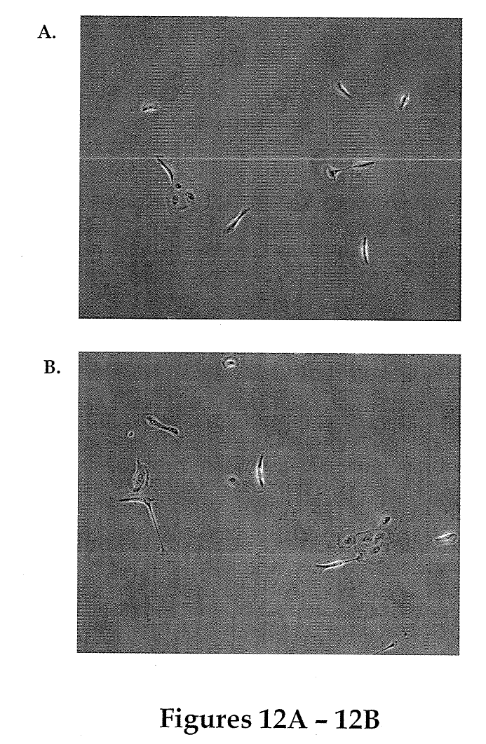

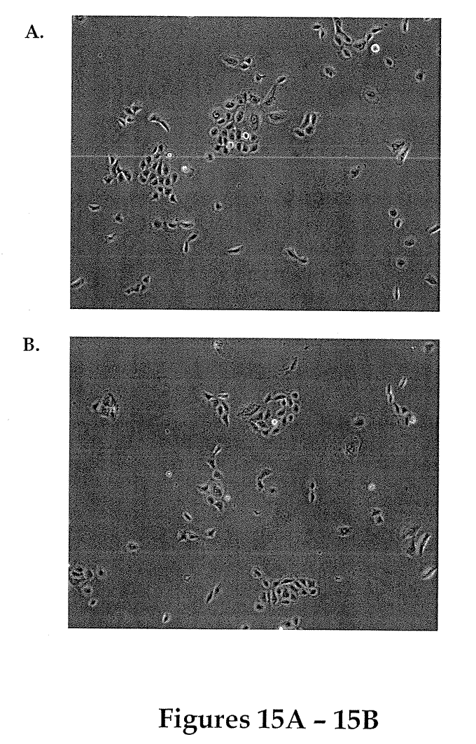

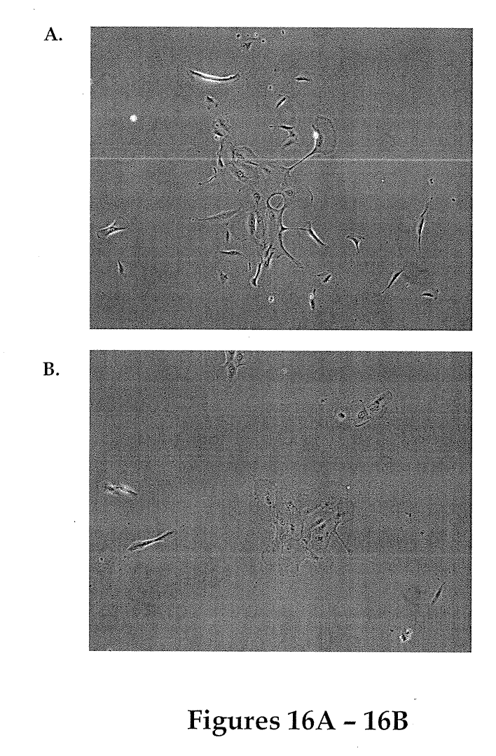

Human keratinocyte cultures seeded at either 5000 cells/well or 10000 cells/well were grown for 3 or 5 days in KBM (FIGS. 3, 7, 11 and 15) or Williams Medium E containing various supplements. Specifically, human keratinocytes were grown on Williams Medium E containing 10% FBS and supplements (insulin (5 mg/L), transferrin (10 mg/L), vitamin D₂ (1 mg/L), linoleic acid-BSA (0.1 mg/L), hydrocortisone (1 mg/L), EGF (10 μg/L), vitamin A (0.115 mg/L), and penicillin (100 U/ml)/streptomycin (100 μg/ml)) (FIGS. 4, 8, 12 and 16).

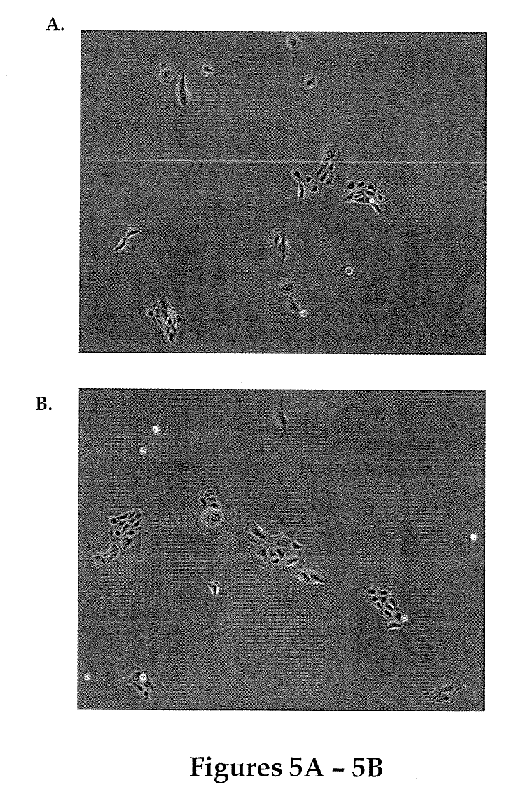

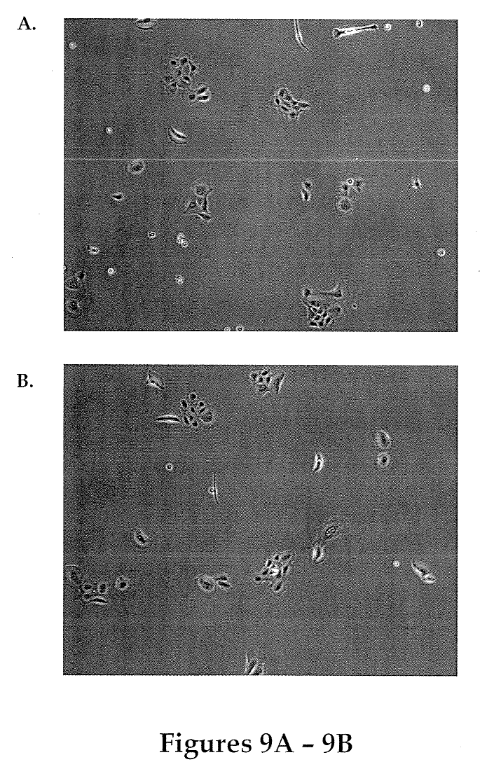

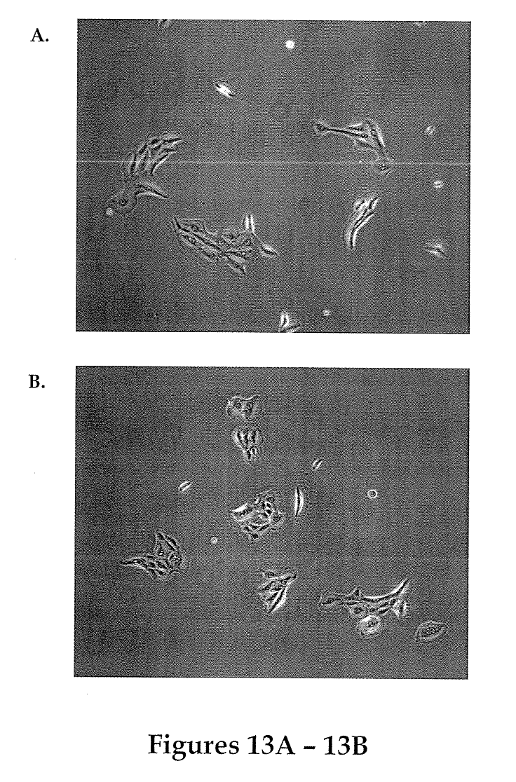

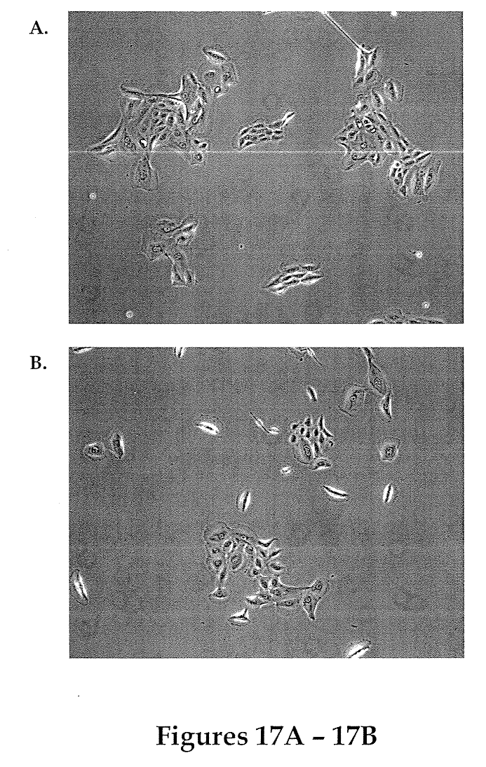

Human keratinocytes were also grown on Super Williams Medium containing supplements (insulin (5 mg/L), transferrin (10 mg/L), vitamin D₂ (1 mg/L), linoleic acid-BSA (0.1 mg/L), hydrocortisone (1 mg/L), EGF (10 μg/L), vitamin A (0.115 mg/L), and penicillin (100 U/ml)/streptomycin (100 μg/ml), delipidized BSA (1.13 g/L), glutamine (2 mM), phosphoethanolamine (5.6 g/L) and ethanolamine (0.122 mg/L)) (FIGS. 5, 9, 13 and 17). This medium is also referred to as Morris 1 Medium.

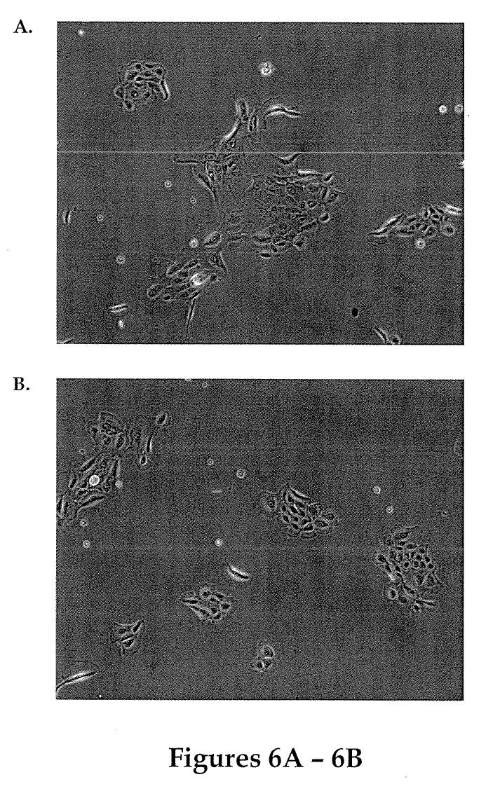

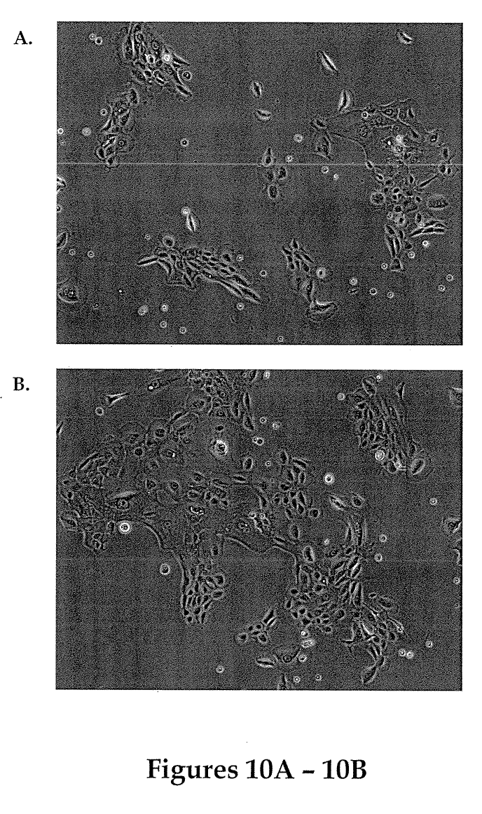

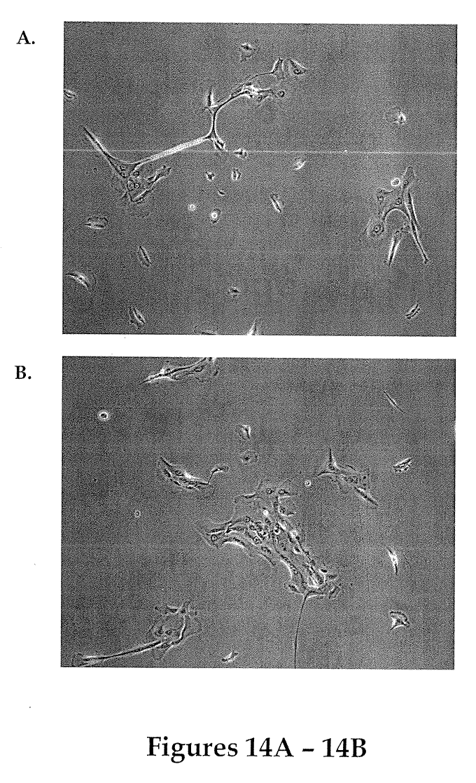

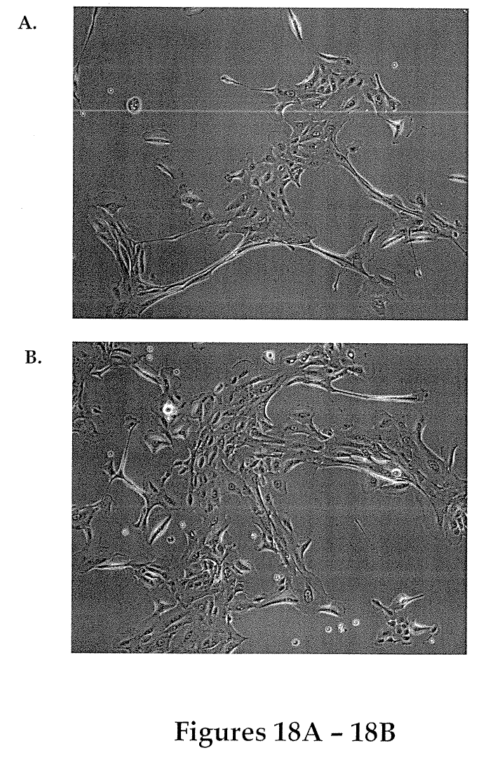

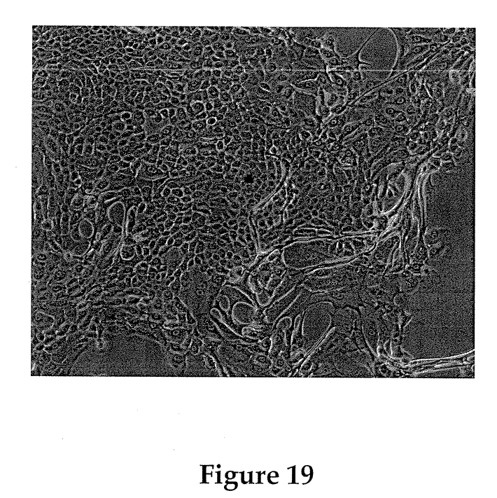

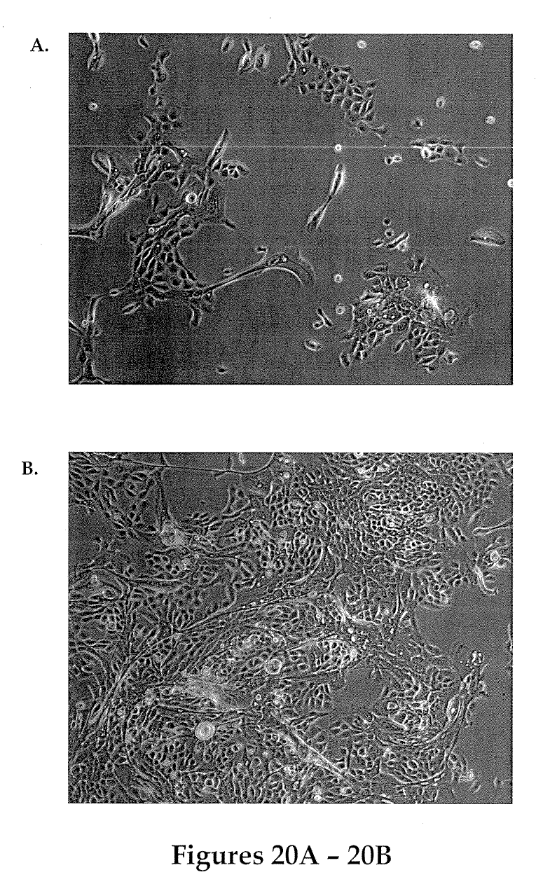

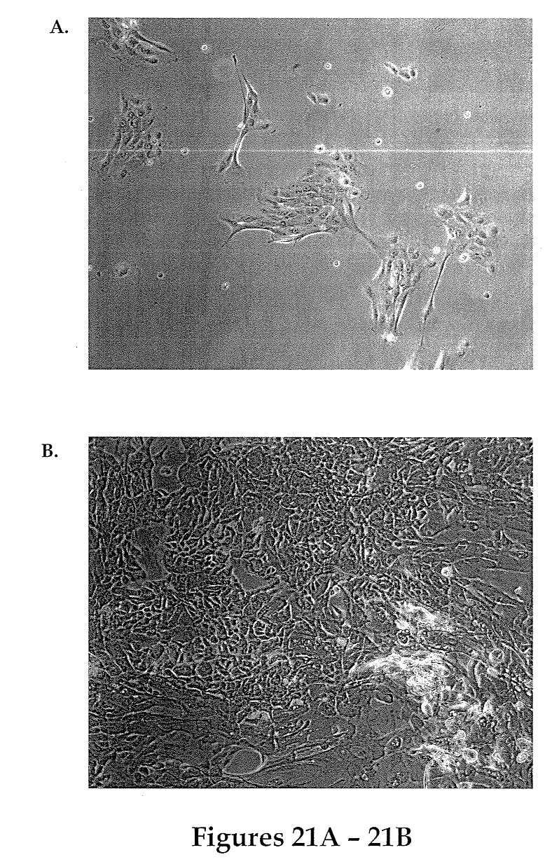

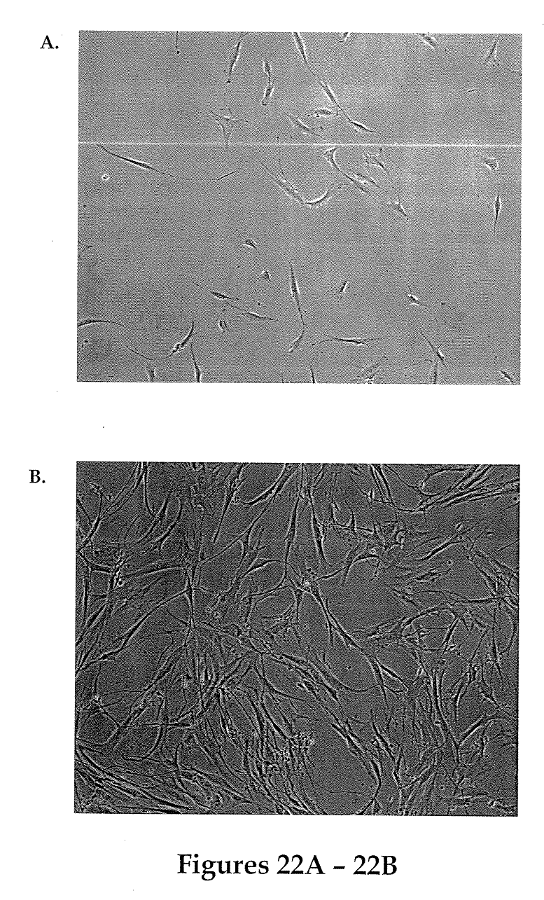

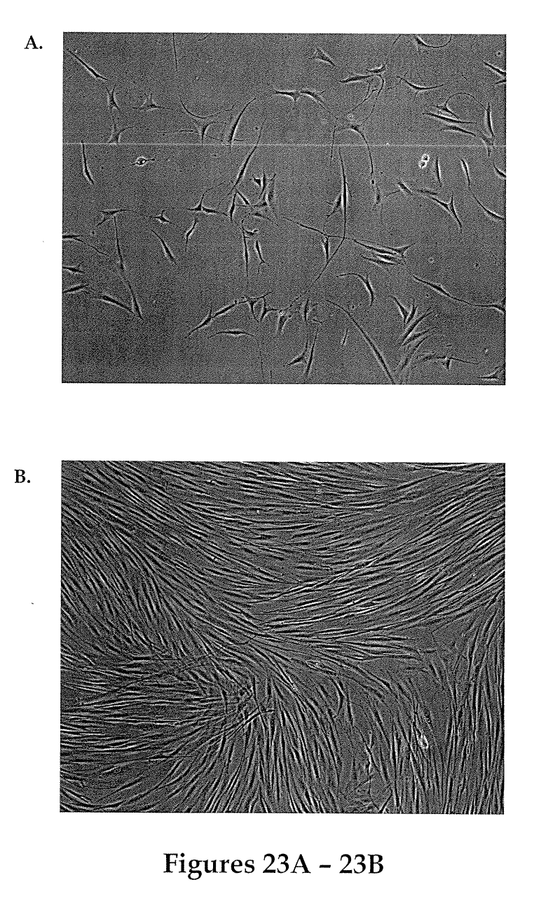

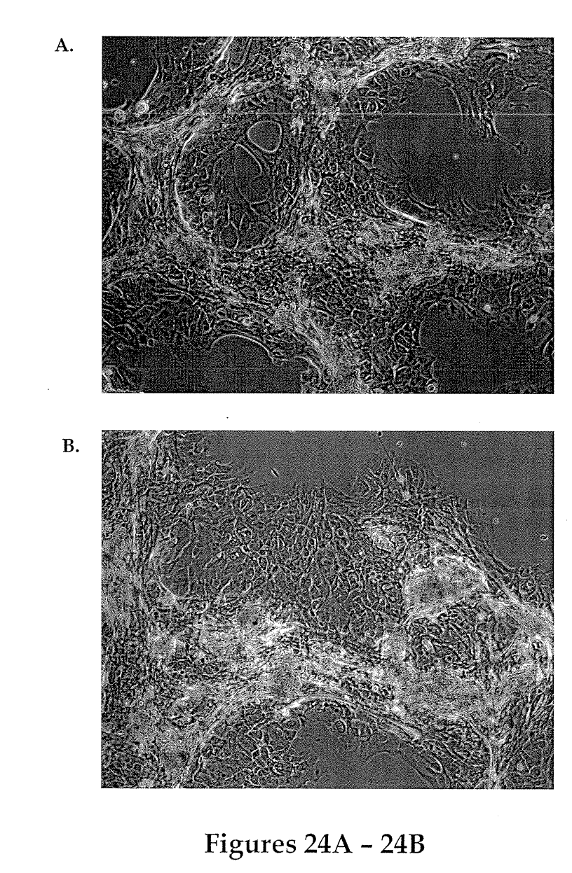

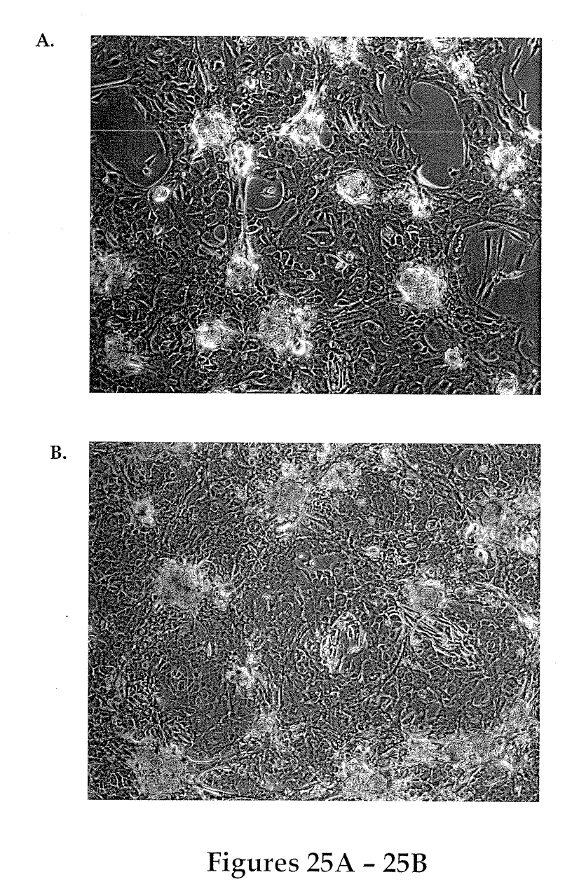

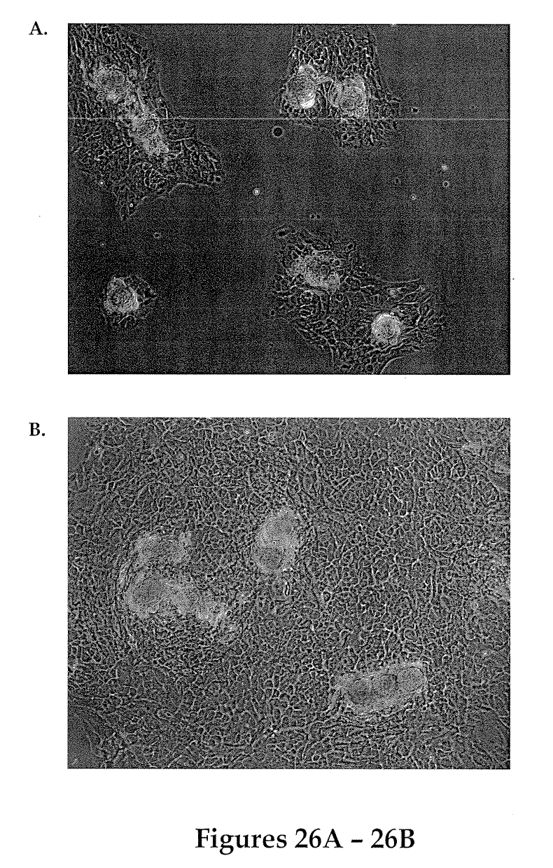

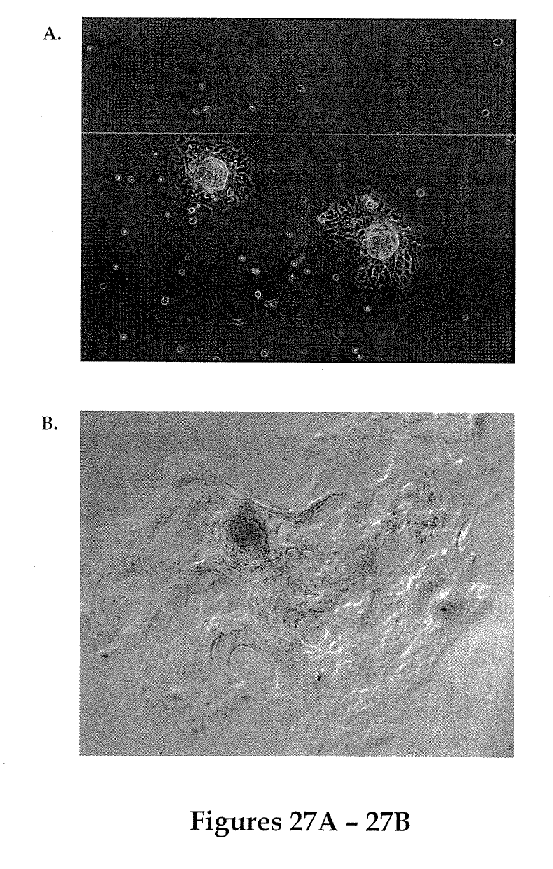

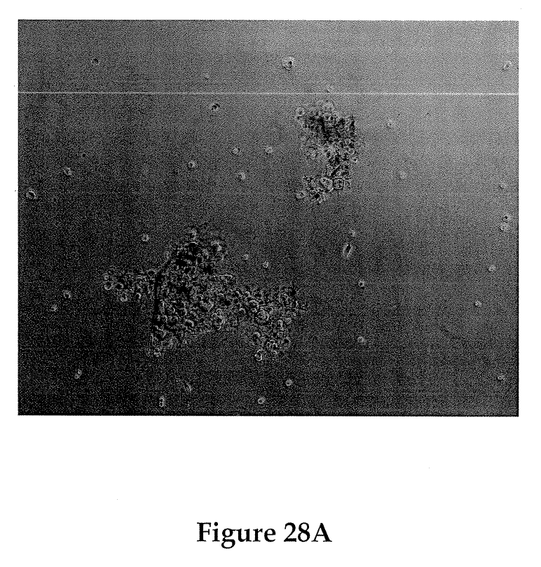

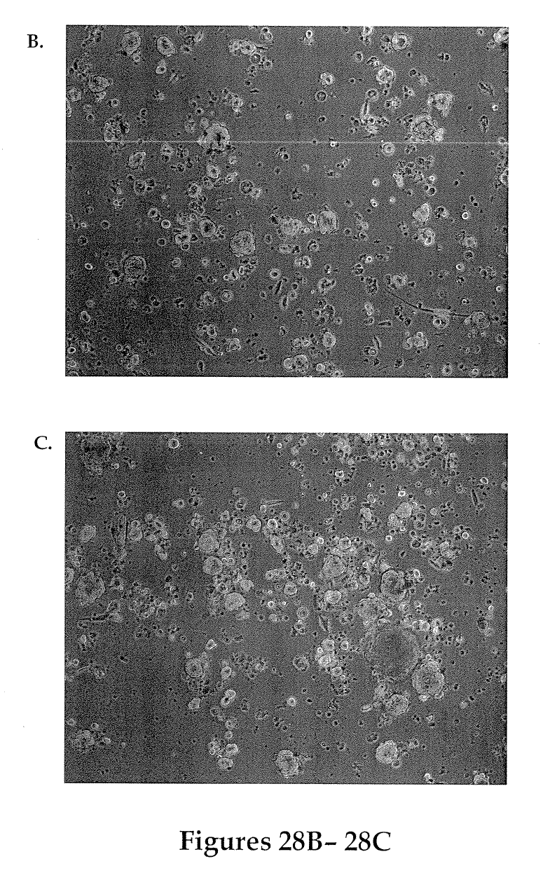

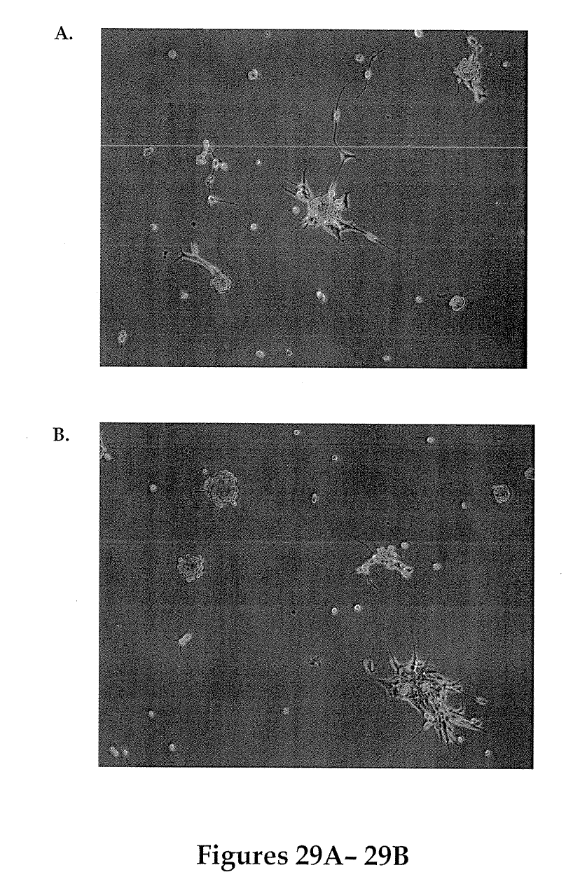

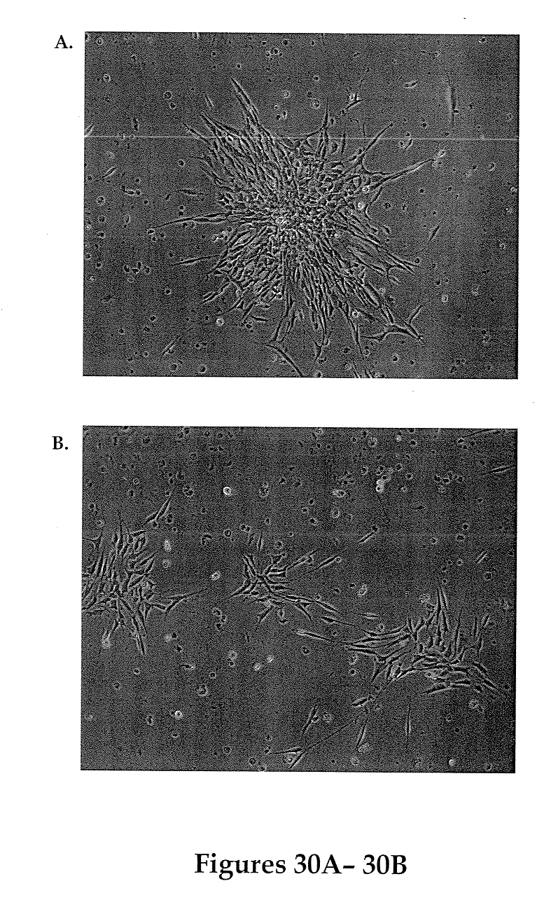

Finally, the growth of human keratinocyte cultures was assessed in off-the-shelf Williams Medium E containing Super Williams supplements (insulin (5 mg/L), transferrin (10 mg/L), vitamin D₂ (1 mg/L), linoleic acid-BSA (0.1 mg/L), hydrocortisone (1 mg/L, EGF (10 μg/L), vitamin A (0.115 mg/L), and penicillin (100 U/ml)/streptomycin (100 μg/ml), delipidized BSA (1.13 g/L), glutamine (2 mM), phosphoethanolamine (5.6 g/L) and ethanolamine (0.122 mg/L)) (FIGS. 6, 10, 14 and 18). This medium represents a chemically-defined medium, which does not contain any unpurified biological components.

While the foregoing invention has been described in some detail for purposes of clarity and understanding, these particular embodiments and examples are to be considered as illustrative and not restrictive. It will be appreciated by one skilled in the art from a reading of this disclosure that various changes in form and detail can be made without departing from the true scope of the invention.

1-14. (canceled)

15. A method for reconstruction of hair follicles, the method comprising
(a) co-culturing epidermal keratinocytes with hair inductive mesenchymal cells, wherein the co-culturing is in the presence of a matrix; and
(b) contacting the co-culture with a chemically defined animal cell culture medium composition comprising:
(i) a synthetic basal medium;
(ii) insulin at a concentration of from about 2.5 mg/L to about 7.5 mg/L;
(iii) transferrin at a concentration of from about 5 mg/L to about 15 mg/L;
(iv) vitamin D₂ at a concentration of from about 0.5 mg/L to about 1.5 mg/L;
(v) linoleic acid-BSA at a concentration of from about 0.05 mg/L to about 0.15 mg/L;
(vi) hydrocortisone at a concentration of from about 0.5 mg/L to about 1.5 mg/L;
(vii) epidermal growth factor (EGF) at a concentration of from about 1 μg/L to about 15 μg/L;
(viii) vitamin A at a concentration of from about 0.0575 mg/L to about 0.1725 mg/L;
(ix) phosphoethanolamine at a concentration of from about 2.8 mg/L to about 8.4 mg/L;
(x) ethanolamine at a concentration of from about 0.061 mg/L to about 0.183 mg/L; and
(xi) delipidized bovine serum albumin (BSA) at a concentration of from about 0.5 g/L to about 1.7 g/L.

16. The method of claim 15, wherein the hair inductive mesenchymal cells comprise keratinocyte stem cells, cells from follicular papillae, sheath cells, or any combination thereof.

17-21. (canceled)

22. A method for identifying whether a test compound is capable of modulating the activity of a hair follicle, the method comprising
(c) contacting with a test compound a hair follicle cultured in a chemically defined animal cell culture medium composition comprising:
(i) a synthetic basal medium;
(ii) insulin at a concentration of from about 2.5 mg/L to about 7.5 mg/L;
(iii) transferrin at a concentration of from about 5 mg/L to about 15 mg/L;
(iv) vitamin D₂ at a concentration of from about 0.5 mg/L to about 1.5 mg/L;
(v) linoleic acid-BSA at a concentration of from about 0.05 mg/L to about 0.15 mg/L;
(vi) hydrocortisone at a concentration of from about 0.5 mg/L to about 1.5 mg/L;
(vii) epidermal growth factor (EGF) at a concentration of from about 1 μg/L to about 15 μg/L;
(viii) vitamin A at a concentration of from about 0.0575 mg/L to about 0.1725 mg/L;
(ix) phosphoethanolamine at a concentration of from about 2.8 mg/L to about 8.4 mg/L;
(x) ethanolamine at a concentration of from about 0.061 mg/L to about 0.183 mg/L; and
(xi) delipidized bovine serum albumin (BSA) at a concentration of from about 0.5 g/L to about 1.7 g/L, and
(d) measuring the activity of the hair follicle in (a) compared to the activity of a hair follicle in the absence of the test compound, so as to identify whether the test compound is capable of modulating the activity of the hair follicle.

23. The method of claim 22, wherein measuring comprises measuring inhibition of hair growth, enhanced hair growth, or loss of hair from the follicle.

24. A method for identifying whether a test compound is capable of modulating hair growth, the method comprising
(a) contacting a test compound with a cultured whole hair follicle, wherein the culturing comprises implanting the follicle into a culture and contacting the implanted follicle with a chemically defined animal cell culture medium composition comprising:
(i) a synthetic basal medium;
(ii) insulin at a concentration of from about 2.5 mg/L to about 7.5 mg/L;
(iii) transferrin at a concentration of from about 5 mg/L to about 15 mg/L;
(iv) vitamin D₂ at a concentration of from about 0.5 mg/L to about 1.5 mg/L;
(v) linoleic acid-BSA at a concentration of from about 0.05 mg/L to about 0.15 mg/L;
(vi) hydrocortisone at a concentration of from about 0.5 mg/L to about 1.5 mg/L;
(vii) epidermal growth factor (EGF) at a concentration of from about 1 μL to about 15 μg/L;
(viii) vitamin A at a concentration of from about 0.0575 mg/L to about 0.1725 mg/L;
(ix) phosphoethanolamine at a concentration of from about 2.8 mg/L to about 8.4 mg/L;
(x) ethanolamine at a concentration of from about 0.061 mg/L to about 0.183 mg/L; and
(xi) delipidized bovine serum albumin (BSA) at a concentration of from about 0.5 g/L to about 1.7 g/L, and
(b) assessing hair growth from the follicle in (a) compared to hair growth from a follicle in the absence of the test compound, so as to identify whether the test compound is capable of modulating hair growth.

25. (canceled)
